(19) United States
(12) Patent Application Publication
(10) Pub. No.: US 2005/0095280 A1
(43) Pub. Date: May 5, 2005

(54) Single-component pH-sensitive liposomes of reduced solid-to-liquid phase transition temperatures

(75) Inventor: Michalakis Savva, Roselle Park, NJ (US)

Correspondence Address: MICHALAKIS SAVVA;APT 38, 240 WEST SUMNER AVENUE, ROSELLE PARK, NJ, 07204, US

(73) Assignee: Michalakis Savva

(21) Appl. No.: 10/686,374

(22) Filed: Oct. 15, 2003

(51) Int. Cl. A61K 9/127; C12N 15/88; A61K 48/00

(52) U.S. Cl. 424/450; 435/458; 514/44; 554/37

(57) ABSTRACT

The current invention relates to the synthesis of novel cationic lipids and their use as delivery vectors for nucleic acids, peptides and other synthetic drugs, in vitro and in vivo. The cationic lipids described herein form stable lamellar structures (liposomes) at physiological pH but destabilize to micelles at acidic and alkaline pH. These structures are characterized of high elasticity, increased fluidity and high transfection activity relative to the corresponding 1,2-dialkyl cationic derivatives and other phospholipids analogues.

### BACKGROUND OF THE INVENTION

Lipid-based drug delivery systems that can be constructed to respond to acidic environments are of great interest to medical sciences, as they can be potentially targeted to release their contents to hyperactive tumor tissues of low pH or to respond by fusion and disruption of primary endosomes, thereby releasing their contents into the cytoplasm.

PH-sensitive liposomes are usually composed of a non-bilayer forming, at physiological temperatures lipid, such as DOPE (1,2-dioleoylphosphatidylethanolamine), and an ionizable, anionic amphiphil, for example, oleic acid or cholesteryl chemisuccinate (CHEMS). When DOPE is mixed with the aforementioned acidic lipids at pH above their pKs, liposomes are formed. Stabilization of DOPE into lamellar (Lₐ) phases is due to the increased size of the amphiphil's polar head group resulting from the increased hydration brought up to, by the attracted (hydrated) counterions. Upon reduction of pH toward the pKa of the anionic amphiphil, fewer counterions are attracted to the polar head group vicinity. As a result, the bilayer vesicles fuse to form reverse hexagonal structures (H₁₁). Few cases of single component pH-sensitive liposomes have been reported in the past. Latest advances in the field include the description of tunable pH-sensitive liposomes composed of mixtures of unsaturated double-chained cationic and anionic lipids. Clearly, all the pH-sensitive bilayer systems described so far, involve mixtures of unsaturated ionizable, anionic phospholipids (CHEMS is the single exemption) that can revert to their most stable H₁₁ phases at acidic pH because of a reduced polar head group area. There has never been any description of a pH-sensitive system where bilayer phases destabilize to micellar or normal hexagonal phases at acidic pH and to reverse hexagonal phases at alkaline pH. The current proposal describes the formation of lamellar phases at physiological pH, made of single type double-chained, ionizable, cationic surfactants bearing saturated acyl chains.

Cationic lipids is a particularly attractive system for gene delivery because it is possible to design and synthesize a wide variety of reagents that depending on their structural features they promote gene expression by affecting one or more of the essential requirements for transfection, i.e. (1) efficient compaction of plasmid DNA (50-80 nm for in vivo and 200-400 nm for in vitro transfections) (2) adhesion of the delivery system onto cell surface (3) lipoplex internalization (4) fusion of the internalized cationic lipid-DNA complex with the endosome/lysosome membrane (5) translocation of the DNA into the nucleus followed by gene expression. Unfortunately, 16 years after pioneering the development of DOTAP and still the design of cationic lipids is largely empirical. Structure-activity relationships remain elusive although several conditions/characteristics of the lipid assemblies in isolation, listed below, have been identified as critical for transfection activity: (1) increased fluidity or low gel-to-liquid phase transition temperature of lamellar assemblies at physiological conditions (2) lamellar-to-reverse hexagonal pH-dependent phase transition. It is suggested that a low gel-to-liquid phase transition temperature of the lipid assembly lowers the activation barrier for cell association and internalization of the lipid-DNA complex. A pH-dependent lamellar-to-reverse hexagonal phase transition promotes fusion of the lipoplex with lysosome membrane allowing the plasmid DNA to escape intact into the cytosol and this is usually the reason of the inclusion of DOPE in the lipoplex formulation.

The present invention relates to new cationic lipids as well as their use for delivering nucleic acid, proteins and other synthetic drugs into cells in vitro and in vivo.

### SUMMARY OF THE INVENTION

The inventor has proposed that cationic lipids of general structure S when dispersed in aqueous solvent can form structures of increased fluidity and elasticity that can facilitate the transfer of nucleic acids, other macromolecules and synthetic drugs into cells.

The structure S of cationic lipids comprises a bifunctional pH-expandable polar head that could be a primary, secondary, tertiary or a quaternary amine or a guanidine group. Cationic lipids of structure S are double-chained derivatives bearing saturated chains that are linked to the propylene synthon at 1 and 3 position. The polar head is linked to the hydrophobic part of the molecule through a connector as shown in structure S.

Assemblies of structure S can associate or encapsulate therapeutic compounds and carry them inside the cells. The enhanced drug delivery potential of these lipids is due to (A) their efficient cell internalization which is in turn due to (1) the high elasticity and (2) the high fluidity that promote fusion of the lipid assemblies with cell membranes and (B) the lysosome escape potential of the carriers promoted by a pH-dependent structure change of the assembly. The structural features and physical properties of cationic lipids of structure S, as described above, obviate the need to use helper lipids such as DOPE in the delivery of therapeutic compounds in vitro and in vivo.

### DESCRIPTION OF FICURES

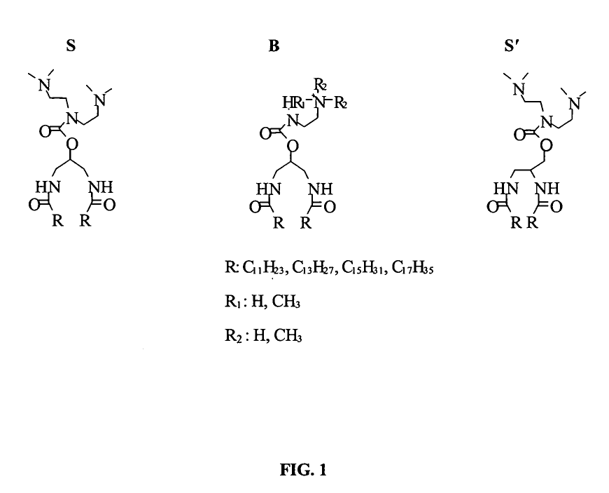

FIG. 1 Structure B is different from structure S in that the polar head is not bifunctional. Structure S′ is the corresponding 1,2-isomers of structure S, that is, the intramolecular distance of the fatty acid chains is reduced as compared to that of structure S lipids.

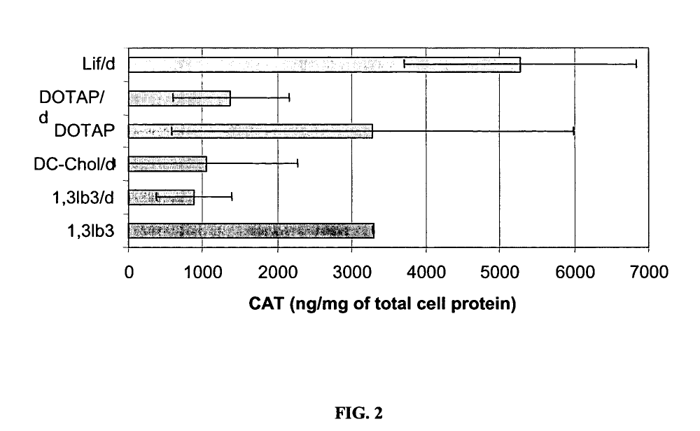

FIG. 2 Transfection activity of 1,3lb3 and other commercially available cationic lipids in B16F0 melanoma cells. Lif/d stands for Lipofectamine/DOPE. The data are the average of six wells two independent experiments (n=3×2).

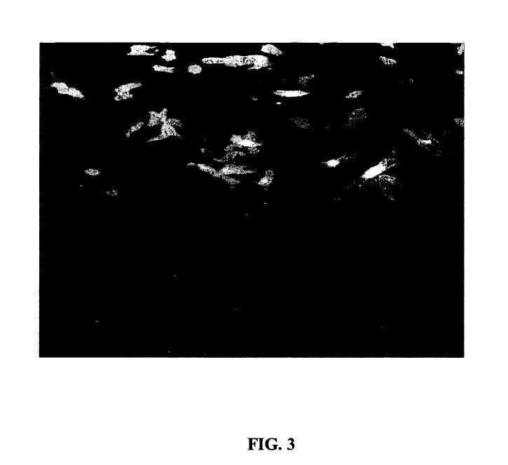

FIG. 3 Transfection activity of 1,3lb3 (in the absence of DOPE) -a dipalmitoyl analogue with fluid lamellar phases at physiological pH and temperature-against B16F0 tumor melanoma cells. The plasmid DNA consisted of an EGFP reporter gene driven by the SV40 early promoter, cloned into a pUC21 vector (CloneTech Labs).

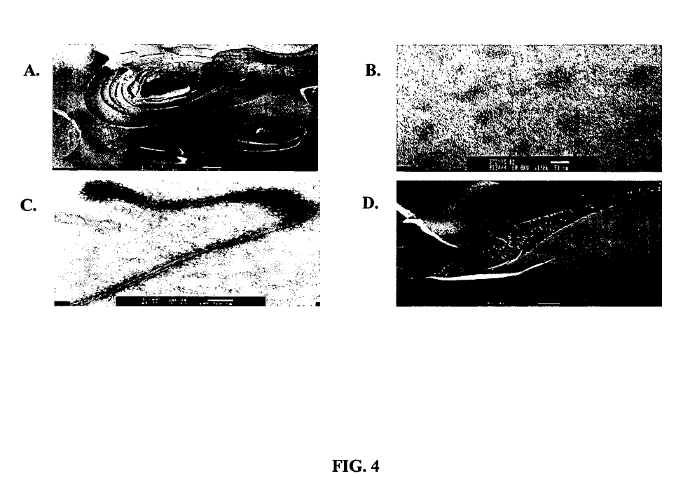

FIG. 4 Transmission (TR) and Freeze Fractured (FF) electron microscopy (EM) of lipid assemblies. A. FFEM of 1,3lb2 pH 7.1 B. TREM (negative stain) of 1,3lb2 pH 5.5 C. TREM (negative stain) of 1,3lmt2 pH 7.1 D. FFEM of 1,3lmt2 pH 5.5.

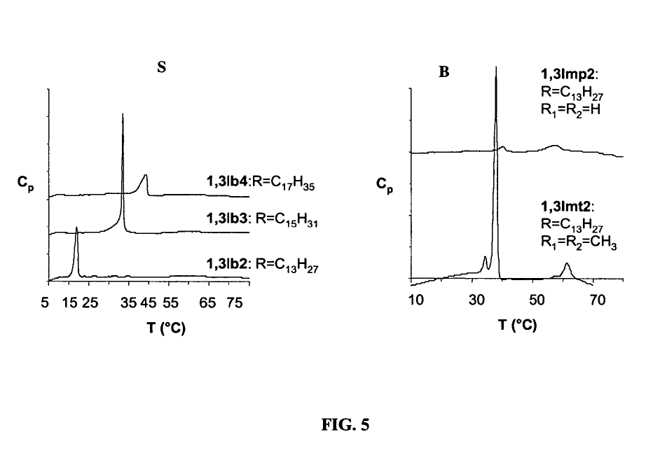

FIG. 5 DSC thermograms of bivalent S and monovalent B amphiphils in 40 mM tris buffer pH 7.1.

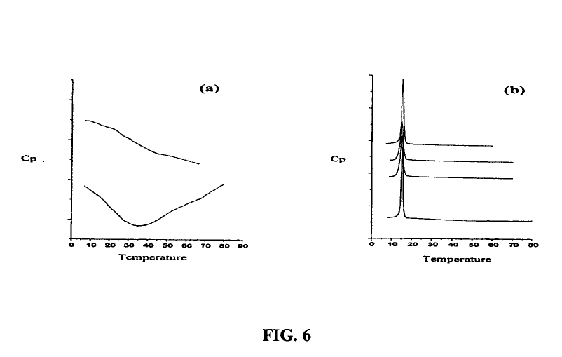

FIG. 6 Successive calorimetric traces of 1,3lb2 in 40 mM (a) MES pH 5.5 and (b) tris pH 7.1.

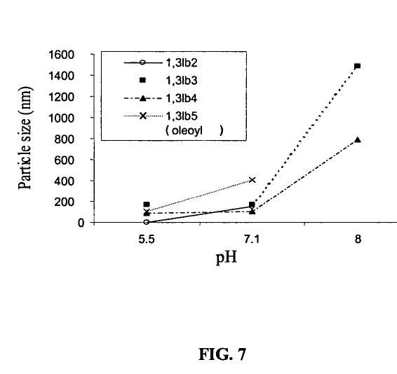

FIG. 7 Mean diameter of S aggregates estimated by Photon Correlation Spectroscopy as a function of pH.

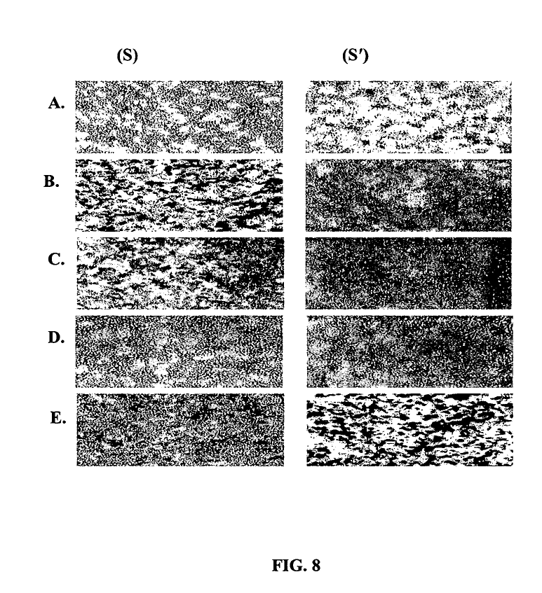

FIG. 8 Transfection activity as tested by X-gal of cationic lipids against B16F0 melanoma cells in the absence of serum. The (+/−) ratio is fixed to 2. A. lb1 B. lb2 C. lb3 D. lb4 E. lb5

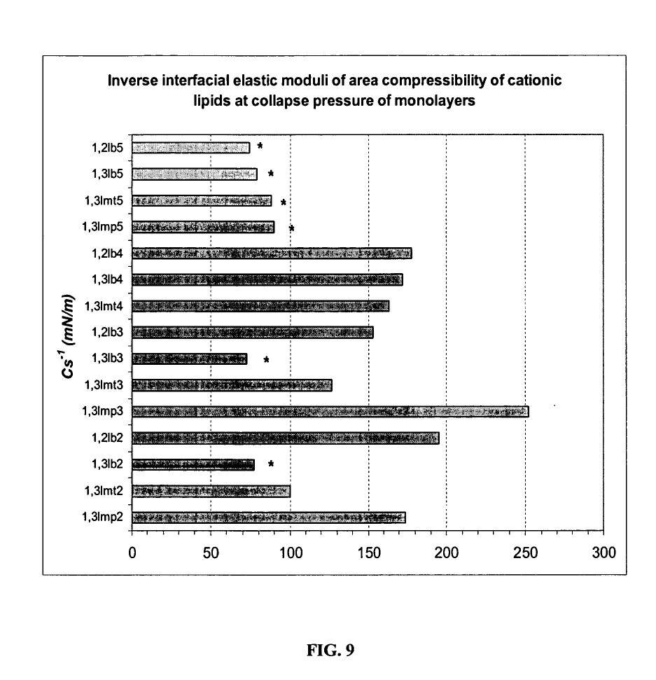

FIG. 9 Isothermal compressibility modulus of cationic lipids at monolayer collapse pressures.

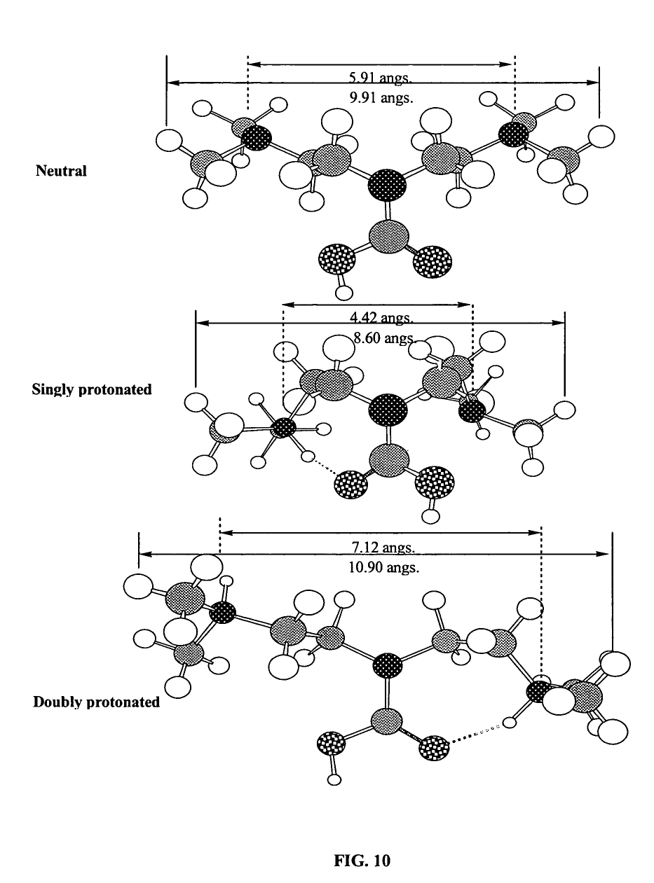

FIG. 10 Optimized geometries of bis-(2-dimethylaminoethyl)-amino carboxylate.

### DESCRIPTION OF THE INVENTION

The present invention describes the creation of novel cationic lipids using a priori design for nucleic acid and other macromolecular drug delivery. The design of the cationic reagents was based on the hypothesis that increasing the distance between the saturated acyl chains of a double-chained cationic lipid would increase the conformational disorder of the lipid, resulting in a bilayer structures when the lipids are dispersed in aqueous solvent, of increased fluidity. The hydrophobic chains interact through van der Waals forces. At low temperatures the acyl chains in phospholipid lamellar assemblies are evidenced to an all-trans configuration. At high enough temperatures, a phase transition is initiated by a trans-gauche rotation of terminal methyl groups followed by a generalized intra chain motion due to rotation of middle C-C bonds. Greater intra molecular spacing of the hydrophobic chains would lower the energetic barrier for an L_{c}-to-Lₐ (solid-to-liquid crystalline) transition.

In order to compensate for the increased “width” of the hydrophobic moiety (1,3-versus 1,2-derivatives) and retain cylindrical geometry of the overall molecule which is an important requirement for liposome formation characterized by increased fluidity, the ionizable, cationic bis-(2-dimethylaminoethyl)amine group was used as the polar part. Expansion of the polar headgroup changes the geometry of the molecule to a cone and that leads to micelle formation. An additional advantage of the polar group is that similar to the hydrophobic chains, it is symmetric. On the other hand, unlike the hydrophobic chains, it is (pH) expandable rendering the lipid molecule and the structure of the assembly pH-sensitive.

Such a design is expected to elicit high transfection efficiency by aiming (1) to improve internalization of lipoplexes by cells and (2) to inhibit lysosome degradation of the therapeutic gene. The current design is expected to improve lipoplex internalization because it forms bilayer structures that have a gel-to-liquid phase transition temperature below 37° C. In addition, a pH-dependent lamellar-to-reverse hexagonal phase transition promotes fusion of the lipoplex with lysosome membrane allowing the plasmid DNA to escape intact into the cytosol. As a result, the presence of DOPE is no longer necessary for transfection activity.

The invention describes a novel way to design and fabricate lipid based delivery systems that are characterized of enhanced drug delivery efficacy due to an increased fluidity and elasticity of the lipid assembly structures. What was known so far, was that increased bilayer fluidity can only be attained with amhiphils composed of unsaturated fatty acid chains. This invention introduces a novel rational approach for the design of fluid bilayers composed of double-chained amphiphils bearing saturated acyl chains as their hydrophobic moiety. There has been many attempts in the past to increase structure fluidity by attaching side-chains in the polar head group or/and the hydrophobic chains. Reduction of the main phase transition temperature was based on intermolecular steric hidrance introduced by the side-groups but as a result lipid polymorphism occurred. What is demonstrated in this description is that by increasing the intramolecular chain distance and by carefully adjusting the polar headgroup dimensions one can design cationic assemblies composed of lipids bearing long saturated chains that are highly elastic and fluid at 37° C.

### EXAMPLE 1

### Synthesis of Bis-(2-dimethylamino-ethyl)-amine (SCHEME I)

To a solution of bis-(2-chloroethyl)-amine HCl (17.1 g; 0.096 mol) in 20 ml water in a 500 ml round bottom flask was added 200 ml dimethylamine (52 g; 1.154 mol). The reaction was stirred for 72 h at room temperature. The reaction was then made alkaline with 6M NaOH (100 ml) and further saturated with anhydrous potassium carbonate. Upon alkalization an oily liquid separates on the upper layer. The mixture was stirred for one half hour and then transferred to a 500 ml separation funnel where it was extracted with 3×150 ml of ethyl ether. The extracts were collected and dried (MgSO₄) overnight. MgSO₄ was removed by suction filtration and the organic solvent was removed under diminished pressure at 45° C. to give a total of 4.6 g of a colorless oil yield (30%). Further purification of the triamine was performed by chromatography as described (Kupchan et al., 1971) with a 50% recovery. MS (FAB & ES) m/z 160.2 [M+H]⁺; ¹H NMR (400 MHz, CDCl₃, 20° C., TMS) δ2.60-2.63 (t, 4H, J=6.2 Hz, NCH₂CH2N(CH₃)₂), 2.31-2.34 (t, 4H, J=6.2 Hz, CH₂N(CH₃)₂), 2.13 (s, 12H, N(CH₃)₂); ¹³C NMR (100 MHz, CDCl₃20° C., TMS) δ59.21 (2CH₂), 47,48 (2CH₂), 45.60 (4CH₃).

Synthesis of representatives of structure S is shown in SCHEME II.

### EXAMPLE 2

### Synthesis of N,N′-ditetradecanoyl-1,3-diaminopropan-2-ol

To a solution of 1,3-diaminopropan-2-ol (0.0133 mol) in 250 ml anh. THF was added TEA (0.0798 mol) and the mixture was stirred at room temperature for 5 min. Dropwise addition of myristoyl chloride (0.0266 mol) was followed over a period of 10 min. The reaction was quenched with addition of 25 ml after 25 min (total of 35 min). After removal of the organic solvent, to the crude material was added 50 ml of distilled water and the mixture was shaked for 5-10 min. Then addition of 250 ml THF was followed to suppress foaming and facilitate filtration. The precipitate was removed by suction filtration and washed several times with ethyl ether before drying for 1 hr in the vacuum oven at 60° C. and air dried for one day. Crystallization of the material from CHCl₃:Et₂O (3:1 v/v) afforded 5.24 g (yield 77%)of N,N′-ditetradecanoyl-1,3-diaminopropan-2-ol as a white crystalline powder; MS (FAB) m/z 511.3 [M+H]⁺; ¹H NMR (400 MHz, CDCl₃, 20° C., TMS) δ6.22 (m, 2H, HNCO), 3.76-3.74 (m, 1H, CH), 3.42-3.24 (m, 4H, CH₂N), 2.23-2.19 (t, 4H, CH₂CO), 1.65-1.60 (m, 4H, CH₂CH₂CO), 1.30-1.26 (coherent peak, 40H, 10(CH₂)₂), 0.90-0.87 (t, 6H, CH₃); ¹³C NMR (100 MHz, CDCl₃, 41° C., TMS) δ176.03 (NHCO), 72.08 (CH), 43.87, 37.85, 33.11, 30.87, 30.84, 30.80, 30.68, 30.51, 26.94, 23.88, 15.29.

### EXAMPLE 3

### N,N′-ditetradecanoyl-1,3-diaminopropane-2-(p-nitrophenyl)carbonate

N,N′-ditetradecanoyl-1,3-diaminopropan-2-ol (0.0098 mol) was suspendeded in 200 ml anh. THF at 50° C. (regulated by an oil bath). To the suspension (very turbid) was added p-nitrophenyl chlorocarbonate (0.0108 mol) followed by a dropwise addition of pyridine (0.0012 mol). After 2.5 h the suspension turns to a clear solution. An additional p-nitrophenyl clhoroformate (0.0108 mol) and pyridine (0.0124 mol) were added and the reaction was stirred for two more hours (total of 4.5 h). The reaction was allowed to cool to room temperature and a thin precipitate, presumably pyridine HCl, was removed by suction filtration. The filtrate was collected and THF was evaporated with the aid of a rotary evaporator. The crude material was dissolved in a minimum quantity of CHCl₃. Unreacted starting material (N,N′-ditetradecanoyl-1,3-diaminopropan-2-ol, insoluble in CHCl₃) was removed by suction filtration. To the filtrate was added more CHCl₃ (150 ml total) and washed once with 150 ml 0.25N HCI (pyridine removal). The aqueous phase was discarded. The organic phase was concentrated and purified by column chromatography (silica gel column 2.8×30 cm), eluting with 100 ml CHCl₃, 100 ml 1%, 2%, 3%, and 200 ml 5%, 7% and 100 ml 12% MeOH/CHCl₃. 100 ml fractions were collected. Fraction 4 and 5 were pooled and concentrated to give a total of 6.14 g (93% yield) of N,N′-ditetradecanoyl-1,3-diaminopropane-2-(p-nitrophenyl)carbonate as a white powder. MS ( ) m/z [M+H]⁺; ¹H NMR (400 MHz, CDCl₃, 20° C., TMS) δ8.29-8.26, 7.43-7.26 (two d, each J=9.1 Hz, 4H,C₆H₄), 6.40-6.37 (m, 2H, HNCO), 4.78-4.76 (m, 1H, CH), 3.60-3.50 (m, 4H, CH₂N), 2.26-2.22 (t, 4H, CH₂CO), 1.65-1.60 (m, 4H, CH₂CH₂CO), 1.28-1.24 (coherent peak, 40H, 10 (CH₂)₂), 0.88-0.85 (t, 6H, CH₃); ¹³C NMR (100 MHz, CDCl₃, 20° C., TMS) δ175.86 (NHCO), 156.32 (OC(O)O), 152.52 (0-C, C₆H₄), 146.28 (C-NO₂, C₆H₄ ), 125.34, 122.84, 76.11 (CH), 38.06, 36.69, 32.51, 31.90, 29.63, 29.47, 29.31, 29.25, 25.69, 22.67, 14.10.

### EXAMPLE 4

### N,N′-ditetradecanoyl-1,3-diaminopropyl-2-carbamoyl-[bis-(2-dimethylaminoethane)] (12lb2)

The synthetic procedure was similar to that described for N,N′-ditetradecanoyl-1,3-diaminopropyl-2-carbamoyl-(N,N-dimethylaminoethane). The yield of the reaction was 68%. MS (FAB) m/z 696.4 [M+H]⁺; ¹H NMR (400 MHz, CDCl₃, 20° C., TMS) δ6.86-6.83 (t, 2H, HNCO), 4.72-4.70 (m, 1H, CH), 3.45-3.30 (m, 8H, (CH₂)₂NC(O)O, CH₂NHC(O)), 2.41-2.35 (m, 4H, (CH₂)₂N), 2.21-2.20 (d, coherent peak, 12H, N(CH₃)₂), 2.19-2.11 (t, 4H, CH₂CO), 1.58-1.53 (m, 4H, CH₂CH₂CO), 1.23-1.20 (coherent peak, 40H, 10(CH₂)₂), 0.84-0.81 (t, 6H, CH₃); ¹³C NMR (100 MHz, CDCl₃, 20° C., TMS) δ174.82 (NHCO), 156.37 (NC(O)O), 73.47 (CH), 59.28, 58.43, 46.98, 46.79, 40.66, 37.92, 33.10, 30.87, 30.84, 30.72, 30.59, 30.57, 30.55, 27.02, 23.89, 15.36.

### EXAMPLE 5

### N,N′-dihexadecanoyl-1,3-diaminopropyl-2-carbamoyl-[bis-(2-dimethylaminoethane)] (12lb3)

MS (FAB) m/z 752.7 [M+H]⁺; ¹H NMR (400 MHz, CDCl₃, 20° C., TMS) δ6.80 (bs, 2H, HNCO), 4.73 (m, 1H, CH), 3.45-3.31 (m, 8H, (CH₂)₂NC(O)O, CH₂NHC(O)), 2.41-2.38 (m, 4H, (CH₂)₂N), 2.23-2.21 (d, coherent peak, 12H, N(CH₃)₂), 2.17-2.13 (t, 4H, CH₂CO), 1.60-1.57 (m, 4H, CH₂CH₂CO), 1.25-1.22 (coherent peak, 48H, 12(CH₂)₂), 0.86-0.83 (t, 6H, CH₃); ¹³C NMR (100 MHz, CDCl₃, 20° C., TMS) δ174.82 (NHCO), 156.41 (NC(O)O), 73.51 (CH), 59.41, 58.50, 47.11, 46.87, 40.70, 38.00, 33.14, 30.93, 30.89, 30.75, 30.62, 30.59, 27.05, 23.93, 15.39.

### EXAMPLE 6

### N,N′-distearoyl-1,3-diaminopropyl-2-carbamoyl-[bis-(2-dimethylaminoethane)] (12lb4)

MS (FAB) m/z 808.6 [M+H]⁺, 780.6 [M⁺-(CH₃)₂]; ¹H NMR (400 MHz, CDCl₃, 20° C., TMS) δ6.90-6.88 (bs, 2H, HNCO), 4.72-4.70 (m, 1H, CH), 3.47-3.29 (m, 8H, (CH₂)₂NC(O)O, CH₂NHC(O)), 2.40-2.36 (m, 4H, (CH₂)₂N), 2.21-2.20 (d, coherent peak, 12H, N(CH₃)₂), 2.15-2.11 (t, 4H, CH₂CO), 1.58-1.55 (m, 4H, CH₂CH₂CO), 1.20 (coherent peak, 56H, 14(CH₂)₂), 0.84-0.81 (t, 6H, CH₃); ¹³C NMR (100 MHz, CDCl₃, 20° C., TMS) δ174.83 (NHCO), 156.40 (NC(O)O), 73.48 (CH), 59.38 (N(CH₃)₂), 58.49 (N(CH₃)₂), 47.07 ((CH₂)₂N), 46.87 ((CH₂)₂N), 40.67, 37.96, 33.13, 30.91, 30.87, 30.74, 30.62, 30.59, 27.03, 23.92, 15.33.

### EXAMPLE 7

### N,N′-dioleoyl-1,3-diaminopropyl-2-carbamoyl-[bis-(2-dimethylaminoethane)] (1,2lb5)

MS (FAB) m/z 804.8 [M+H]⁺; ¹NMR (400 MHz, CDCl₃, 20° C., TMS) δ6.83 (bs, 2H, HNCO), 5.36-5.31 (m, 4H, CH=CH), 4.75-4.73 (m, 1H, CH), 3.56-3.52 (m, 4H, (CH₂)₂NC(O)), 3.37-3.29 (m, 4H, CH₂NHC(O)), 2.44-2.41 (m, 4H, (CH₂)₂N), 2.26-2.24 (d, coherent peak, 12H, N(CH₃)₂), 2.19-2.15 (t, 4H, CH₂CO), 2.00-1.95 (m, 8H, CH₂CH=CHCH₂), 1.61 (m, 4H, CH₂CH₂CO), 1.29-1.25 (coherent peak, 40H, 10(CH₂)₂), 0.88-0.84 (t, 6H, CH₃); ¹³C NMR (100 MHz, CDCl₃, 20° C., TMS) δ174.81 (NHCO), 156.38 (NC(O)O), 131.40-130.52 (C=C), 73.44 (CH), 65.34, 59.28, 58.46, 46.96, 46.77, 40.58, 37.86, 33.86, 33.77, 33.70, 30.94, 30.90, 30.84, 30.79, 30.71, 30.66, 30.62, 30.53, 30.50, 30.40, 30.38, 30.32, 30.27, 30.12, 28.40, 28.38, 27.65, 23.87, 15.34.

### EXAMPLE 8

Transfection activity of dipalmitoyl representative of structure S, 13lb3 is shown in FIGS. 2-3. The lipid mediates efficient transfection activity even in the absence of helper lipid DOPE.

### EXAMPLE 9

Structure S amphiphiles form bilayers structures (liposomes) at physiological conditions (FIG. 4A) as opposed to the formation of rod-structures by structure B amphiphiles (FIG. 4C) verifying that bifunctional polar head is a requirement for liposome formation.

### EXAMPLE 10

DSC studies shown in FIG. 5 verify the increased fluidity of structure S assemblies as opposed to the polymorphic nature of the structure B assemblies.

### EXAMPLE 11

FIG. 4B and FIG. 6, verify the pH-sensitivity of structure S self assemblies. At acidic pH (5.5) all liposomes were destroyed and only small size micelles can be detected. The pH-dependent topological shifting of structure S assemblies is also verified by dynamic light scattering. Particle size increases with increasing pH (FIG. 7).

### EXAMPLE 12

FIG. 8 shows that the 1,3 amphiphiles of structure S can mediate high levels of gene expression as compared to no activity of the corresponding 1,2 amphiphiles of structure S′. Structure S is characterized with reduced main phase transition temperature, thus increased fluidity and high elasticity (FIG. 9). Thus, increasing the distance between the saturated acyl chains of a double-chained cationic lipid (structure S) increases the conformational disorder of the lipid, resulting in a bilayer of increased fluidity.

### EXAMPLE 13

The pH-dependent dimension of the bifunctional polar head group was also simulated using semi-empirical quantum mechanical methods (FIG. 10).

1. Aqueous lipid dispersions made by double-chained cationic lipids that have a bifunctional polar head and the two hydrophobic chains composed of linear alkyl (saturated) hydrocarbons are at position 1 and 3 as shown below, for nucleic acid, peptide and other synthetic molecule drug delivery.

2. The delivery system of claim 1, and any other ingredient that is added to the system of claim 1 for activity or other technical reasons. 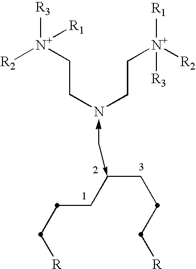
Structure S of Cationic Lipids:
R=C₁₁H₂₃, C₁₃H₂₇, C₁₅H₃₁, C₁₇H₃₅ (linear chains)
R₁=H, CH₃, —C(NH₂)═NH
R₂=H, CH₃
R₃=H, CH₃ [chemical structure]
